(19) United States
(12) Patent Application Publication
(10) Pub. No.: US 2014/0179719 A1
(43) Pub. Date: Jun. 26, 2014

(54) NOVEL AMORPHOUS SOLID DISPERSIONS OF VALGANCICLOVIR HYDROCHLORIDE

(71) Applicant: SREEDHAR CHEEKOORI, TORONTO (CA)

(72) Inventor: SREEDHAR CHEEKOORI, TORONTO (CA)

(21) Appl. No.: 13/727,560

(22) Filed: Dec. 26, 2012

(51) Int. Cl. A61K 9/10

(52) U.S. Cl. CPC A61K 9/10
USPC 514/263.38

(57) ABSTRACT

Disclosed are novel amorphous solid dispersion formulations comprising valganciclovir hydrochloride.

### FIELD OF THE INVENTION

The present invention relates to amorphous solid dispersion formulations comprising valganciclovir hydrochloride and useful in the treatment of viral diseases, in particular used in the treatment of cytomegalovirus (CMV) infections.

### BACKGROUND OF THE INVENTION

Cytomegalovirus (CMV) is a common infectious complication in patients with acquired immunodeficiency syndrome (AIDS), including solid organ transplant (SOT) recipients and is a significant cause of morbidity and mortality. Ganciclovir has been well established for the prevention and treatment of CMV infection and disease. Long-term intravenous (IV) administration as prophylaxis, however is impractical and the low bioavailability (6-10%) of the oral formulation limits the use.

Valganciclovir, the oral valine ester prodrug of ganciclovir, overcomes the limitations of poor oral bioavailability of ganciclovir and is a convenient alternative to IV administration. Valganciclovir is well absorbed from the gastrointestinal tract and rapidly converted to ganciclovir in the intestinal wall and liver. The bioavailability of ganciclovir following oral administration of valganciclovir is around 60% compared to that achieved with 5 mg/kg IV ganciclovir and approximately 1.7 times that achieved with oral ganciclovir 1000 mg three times a day.

Valganciclovir hydrochloride sold as VALCYTE® and with a chemical name L-valine, 2-[(2-amino-1,6-dihydro-6-oxo-9H-purin-9-yl)-methoxy]-3-hydroxypropyl ester, monohydrochloride. The molecular formula is C₁₄H₂₂N₆O₅ HCl with molecular weight of 390.83, and is represented by structural Formula below:

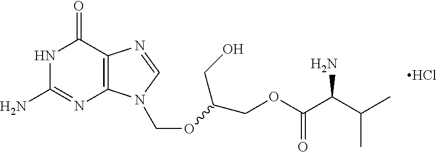 [SMILES: CC(C)[C@H](N)C(=O)OCC(CO)OCn1cnc2c(=O)[nH]c(N)nc21 ; Cl]

U.S. Pat. No. 6,083,953 discloses process of preparing crystalline valganciclovir hydrochloride. The patent also teaches that valganciclovir hydrochloride was developed to improve the bioavailability of gancilcovir, which has a poor bioavailability (6%) when administered orally.

United States Patent Application Nos. 2007/0129385, 2009/0062538, 2010/0298564 discloses process to prepare amorphous form of valganciclovir hydrochloride.

As amorphous forms are thermodynamically unstable relative to the corresponding crystal forms, it is well known that amorphous form would revert back to the stable crystalline form. This usually occurs during storage at various humidities and temperatures. Therefore, it is necessary to inhibit crystallization of amorphous forms over the period of product storage and maintain sufficient level of super saturation upon oral administration without crystallization.

It has now been found that certain polymers are useful for preparing solid dispersions of amorphous valganciclovir hydrochloride having significant solubility improvements over conventional formulations and also possessing significant physical stability improvements over amorphous drug substance alone. The present invention provides such stable amorphous dispersions of valganciclovir hydrochloride with improved solubility and stability.

### SUMMARY OF THE INVENTION

In the first aspect, this invention provides stable amorphous solid dispersion of the active agent valganciclovir hydrochloride. The present invention also provides processes for preparing and compositions comprising the amorphous solid dispersions of the instant invention, and to methods of use thereof.

Another embodiment of the present invention is a method for treating cytomegalovirus (CMV) in patients with acquired immunodeficiency syndrome (AIDS), and for the prevention of CMV disease in kidney, heart, and kidney-pancreas transplant patients who are at high risk.

According to the present invention, solid dispersions of amorphous valganciclovir hydrochloride have significant solubility and physical stability improvements over amorphous drug substance alone.

### DETAILED DESCRIPTION OF THE INVENTION

Cytomegalovirus (CMV) is infection occurs when the human immune defenses are weak and can attack several parts of the body causing severe damage. The most common illness is retinitis (cell death in retina) resulting in blindness if untreated.

Valganciclovir hydrochloride sold as VALCYTE® has been used for the treatment of CMV in patients with weak immune system.

The object of present invention is to obtain an amorphous solid dispersion of valganciclovir hydrochloride with good physical stability, enhanced dissolution and oral bioavailability.

Another object of present invention is an amorphous solid dispersion comprising amorphous valganciclovir hydrochloride and a stabilizing polymer, where valganciclovir hydrochloride is present in substantially amorphous solid state form, that is at least 80% of valganciclovir hydrochloride in the dispersion is in an amorphous form, preferably 85%, more preferably at least 90% and most preferably at least 95% of valganciclovir hydrochloride in the dispersion is in amorphous form.

The amount of valganciclovir hydrochloride in the amorphous solid dispersion of the present invention ranges from about 1% to 30% by weight relative to the stabilizing polymer. In a preferred embodiment, the amount of valganciclovir hydrochloride ranges from 10% to 25%, more preferably from 5% to about 20% by weight relative to the stabilizing polymer.

Stabilizing polymers of the present invention includes to anyone of hydroxypropyl methylcellulose phthalate (HPMCP), cellulose acetate phthalate (CAP), hydroxypropyl methyl cellulose acetate succinate (HPMCAS) and polymeric polymethacrylates, such as EUDRAGIT® L 100 and mixtures thereof. Most preferred stabilizing polymers of the present invention include, hydroxypropyl methylcellulose phthalate, cellulose acetate phthalate, and polymeric polymethacrylate.

The amorphous solid dispersions of valganciclovir hydrochloride of the present invention prepared by conventional techniques known for example and not limited to spray drying, melt extrusion, freeze drying, rotary evaporation and drum drying. Preferred method is use of spray drying. Suitable solvents for these methods may be selected from water, alcoholic solvents, ketones, esters, ethers, halogenated solvents, hydrocarbons, nitriles, water aprotic polar solvents or mixtures thereof. Alcohol solvents such as for example methanol, ethanol, denatured spirit, n-propanol, isopropanol, n-butanol, isobutanol, and t-butanol; ketonic solvents such as acetone, propanone, and 2-butanone; halogenated solvents, such as dichloromethane, 1,2-dichloroethane, chloroform, and carbontetrachloride; ester solvents, such as ethyl acetate, n-propyl acetate, isopropylacetate and n-butyl acetate and the like; ether solvents such as for example dimethylether, diethylether, methyltertiarybutylether, ethylmethylether, diisopropylether, tetrahydrofuran, and dioxane. The hydrocarbon may be any solvent from this class such as for example toluene, xylene, cyclohexane, n-hexane, and n-heptane. The nitrile solvents may include acetonitrile, and propionitrile; aprotic polar solvents, such as N,N-dimethylformide (DMF), Dimethylsulfoxide (DMSO), and N,N-dimethylacetamide (DMA) or mixtures thereof.

In addition, amorphous solid dispersion of valganciclovir hydrochloride that are contemplated by the present invention may further included are pharmaceutically acceptable excipients. Examples of pharmaceutically acceptable excipients include, but are not limited to binders, fillers or diluents, lubricants, glidants and disintegrants. A combination of excipients may also be used. The amount of excipient(s) employed will depend upon how much active agent is to be used. One excipient can perform more than one function.

Binders include, but are not limited to, starches such as potato starch, wheat starch, corn starch; microcrystalline cellulose such as products known under the registered trademarks Avicel, Filtrak, celluloses such as hydroxypropyl cellulose, hydroxyethyl cellulose, ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al, Ca, Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants include, but are not limited to: alginic acid, carboxymethylcellulose calcium, carboxymethylcellulose sodium, croscarmellose sodium, crospovidone, guar gum, magnesium aluminium silicate, sodium alginate, sodium starch glycolate and starches and other materials known to one of ordinary skill in the art and combinations thereof.

The pharmaceutical compositions of the present invention are generally administered to humans, in the form of, for example, a hard or soft gelatin capsule, a tablet, a caplet, pills, granules or a suspension.

Preferred unit dosages of the pharmaceutical compositions of this invention typically contain from 100 mg to 1000 mg of the novel amorphous solid dispersion of valganciclovir hydrochloride. Most preferably 400 mg to 950 mg.

The following examples will further illustrate the invention, without, however, limiting it thereto.

### EXAMPLES

### Example 1

Preparation of amorphous solid dispersion of valganciclovir hydrochloride with hydroxypropyl methyl cellulose phthlate:

Valganciclovir hydrochloride (1.0 g) and hydroxypropyl methyl cellulose (4.0 g) was dissolved in methanol (40 ml), and the solution was fed into a spray drier through a peristaltic pump at 10 ml/min. The temperature at the inlet of the drying chamber was maintained at 50° C. The solvent was removed to provide amorphous solid dispersion.

### Example 2

Preparation of amorphous solid dispersion of valganciclovir hydrochloride with hydroxypropyl methyl cellulose phthlate:

Valganciclovir hydrochloride (1.0 g) and hydroxypropyl methyl cellulose (4.0 g) was dissolved in methanol (40 ml). The resulting solution was vaccum evoparated at 60° C. to 70° C. resulting in the amorphous solid dispersion.

1) An amorphous solid dispersion comprising substantially amorphous valganciclovir hydrochloride, a stabilizing polymer and an excipient.

2) The amorphous solid dispersion according to claim 1, wherein said stabilizing polymer is one or more polymers selected from the group consisting of cellulose acetate phthalate, hydroxypropyl methyl cellulose acetate succinate, polymeric polymethacrylate and a hydroxypropyl methylcellulose phthalate or in combinations thereof.

3) The amorphous solid dispersion according to claim 2, wherein said stabilizing polymer is cellulose acetate phthalate.

4) The amorphous solid dispersion according to claim 2, wherein said stabilizing polymer is, hydroxypropyl methyl cellulose acetate succinate.

5) The amorphous solid dispersion according to claim 2, wherein said stabilizing polymer is hydroxypropyl methylcellulose phthalate

6) The amorphous solid dispersion according to claim 2, wherein said stabilizing polymer is polymeric polymethacrylate.

7) The amorphous solid dispersion according to claim 5, wherein the polymeric polymethacrylate is EUDRAGIT® L 100.

8) The amorphous solid dispersion according to claim 1, wherein valganciclovir hydrochloride is present in an amount of from about 1% to about 30% by weight relative to the weight of the stabilizing polymer.

9) The amorphous solid dispersion according to claim 8, wherein valganciclovir hydrochloride is present in an amount of from about 5% to about 20% by weight relative to the weight of the stabilizing polymer.

10) The amorphous solid dispersion according to claim 1, wherein at least 80% valganciclovir hydrochloride is in amorphous form.

11) The amorphous solid dispersion according to claim 1, wherein at least 85% valganciclovir hydrochloride is in amorphous form.

12) The amorphous solid dispersion according to claim 1, wherein at least 90% valganciclovir hydrochloride is in amorphous form.

13) The amorphous solid dispersion according to claim 1, wherein at least 95% valganciclovir hydrochloride is in amorphous form.

14) A pharmaceutical composition according to claim 1 for the treatment or prevention of viral diseases.
